(19) United States
(12) Patent Application Publication
(10) Pub. No.: US 2008/0306486 A1
(43) Pub. Date: Dec. 11, 2008

(54) Surgical Alignment Guide

(76) Inventor: Robert Lye, New South Wales (AU)

Correspondence Address: HENRICKS SLAVIN AND HOLMES LLP;SUITE 200, 840 APOLLO STREET, EL SEGUNDO, CA, 90245, US

(21) Appl. No.: 12/097,756

(22) PCT Filed: Nov. 16, 2006
(86) PCT No.: PCT/AU06/01716

§ 371 Date: Jun. 17, 2008

(30) Foreign Application Priority Data
Nov. 18, 2005 (AU) 2005906437

(51) Int. Cl. A61B 17/58; A61F 2/32

(52) U.S. Cl. 606/91; 623/22.12; 623/22.21

(57) ABSTRACT

A surgical alignment guide for hip replacement or revision surgery includes a support (12) for a prosthetic acetabular cup, the support being connected to one end of a carrier shaft (10). The support (12) has a bore (14) angled relative to the shaft so that a prosthetic acetabular cup secured to the support (12) has the correct orientation. An arm (26) extends from the shaft (10) at a position spaced from the support (12), and an adjustment arrangement (17, 21) is associated with the shaft (10) to enable adjustment of the angular position of the bore (14) with respect to the arm (26). In one form, an outer end of the arm (26) is connected to a gauge (28) which provides an alignment orientation indication of the cup support (12) and a prosthetic acetabular cup attached thereto. The adjustment arrangement may have two radial fingers (17) extending from the shaft (10) and engagable with teeth (24) associated with the arm (26).

### FIELD OF THE INVENTION

This invention relates to a surgical alignment guide and relates particularly to an alignment guide for aligning a prosthetic acetabulum cup in the reamed acetabulum of a patient undergoing hip replacement surgery.

### BACKGROUND OF THE INVENTION

In International Patent Application PCT/AU02/01482 dated 1 Nov. 2002, an alignment handle for use in hip replacement surgery is described. The described alignment handle includes a body with a gauge at one end thereof and a connector at the other end. The gauge, preferably a plumb bob or the like, provides a visual indication when the alignment handle is in a predefined orientation with respect to at least a first plane in a referential system.

That specification discloses an alignment handle which is able to be connected to a left hand or a right hand prosthetic acetabular cup support depending on whether the hip, the subject of the surgery, is the left hip or the right hip of the patient.

However, the provision of selectable left hand and right hand cup holders for use with the alignment handle requires a decision as to which cup holder is to be used for particular surgery giving rise to the potential for an error to occur.

Further, the left hand and right hand cup holders are, when assembled with the alignment handle, fixed in the appropriate position relative to the alignment handle. In many cases, however, the desired angle of the cup holder relative to the alignment handle may be slightly different to the fixed angle defined by the alignment handle and cup holder.

For most patients, a normal abduction angle is 45°, while the anteversion is normally about 15°. Some patients, however, require slightly different angular orientation of the acetabular cup.

Some prior art techniques for hip replacement surgery require the surgeon to align a reaming tool and a prosthetic acetabular cup with a patient's acetabulum by eye. This technique entails a risk of misalignment of the prosthetic acetabular cup as impacted into the patient's pelvis.

Even when using the alignment handle as described in our earlier patent application, a misalignment may occur in relation to a patient who requires an abduction angle or anteversion which is different from the normal.

It is therefore desirable to provide an alignment guide which obviates at least some of the disadvantages of aligning a reaming tool and a prosthetic acetabular cup by eye.

It is also desirable to provide an improved alignment guide which facilitates alignment of a prosthetic acetabular cup with selected abduction and anteversion angles.

It is also desirable to provide an alignment guide which is relatively simple to manufacture and use.

It is also desirable to provide an alignment guide which is simple to adjust to the desired position.

It is also desirable to provide a surgical alignment guide for hip replacement surgery to facilitate alignment of a prosthetic acetabular cup on a variety of patients having an abduction angle and/or an anteversion which varies from normal.

### SUMMARY OF THE INVENTION

According to one aspect of the invention there is provided a surgical alignment guide for hip replacement or revision surgery comprising a prosthetic acetabular cup support connected to one end of an elongated carrier, said support having a bore to receive a connector to releasably secure a prosthetic acetabular cup thereto in a predetermined orientation, said bore extending at a predetermined angle relative to the carrier, an arm extending from the carrier at a position spaced from the support, and adjustment means associated with the elongated carrier whereby the angular position of the bore is variable with respect to the arm.

Preferably, the elongated carrier comprises a rod or shaft having the cup support fixed to one end thereof.

The cup support may comprises a boss fixed to the end of the rod or shaft, as by welding, so as to extend at a fixed angle, such as 45° to the shaft axis. In one embodiment, the bore through the boss is axial, and has an enlarged diameter at least one, preferably both ends thereof. The enlarged bore section at a lower end is adapted to receive a spacer of a predetermined thickness to facilitate correct engagement of different sizes of prosthetic acetabular cups to the cup support boss.

In some preferred embodiments, the elongated carrier includes a handle portion at the end spaced from the cup support. The handle portion may be tubular and coaxial with the elongated carrier, and being mounted thereto by a threaded connection.

The elongated carrier, in these embodiments, engages through an opening in the arm, at one end thereof, and is rotatable about its axis relative to the arm. A locking screw is provided in said one end of the arm, the locking screw being adapted to engage within one of at least two detents formed in the elongated carrier. The engagement of the locking screw with one or other of the detents fixes the relative angular orientation of the cup support bore, and, therefore, the angular position of a prosthetic acetabular cup mounted thereto, in relation to the arm position. The locking screw may engage axially in said one end of the arm.

The outer end of the arm may be adapted to be connected to a gauge which provides an alignment orientation indication of the cup support and a prosthetic acetabular cup attached thereto. Such a gauge may be a plumb bob gauge or any other form of gauge which provides an appropriate indication of angular orientation relative to a reference, such as a reference plane or line.

In another embodiment, the adjustment means associated with the elongated carrier comprises one, preferably two radial fingers extending from the elongated carrier. The fingers are provided with teeth on a radial surface, the teeth being adapted to engage corresponding teeth associated with the arm. With this arrangement, the angular orientation of the bore is able to be adjusted through a range of different positions by varying the engagement of the respective teeth.

In order that the invention is more readily understood, two embodiments thereof will now be described with reference to the accompanying drawings.

### DESCRIPTION OF THE DRAWINGS

FIG. 1 is a perspective view of a first embodiment of the invention;

FIG. 2 is a partially exploded view of the alignment guide of FIG. 1;

FIG. 3 is a detailed view of one form of adjusting means for a prosthetic acetabular cup support;

FIG. 4 is a detailed view of a complimentary part of the adjusting means; and

FIG. 5 is an exploded view of a surgical alignment guide in accordance with a second embodiment of the invention.

### DESCRIPTION OF THE PREFERRED EMBODIMENTS

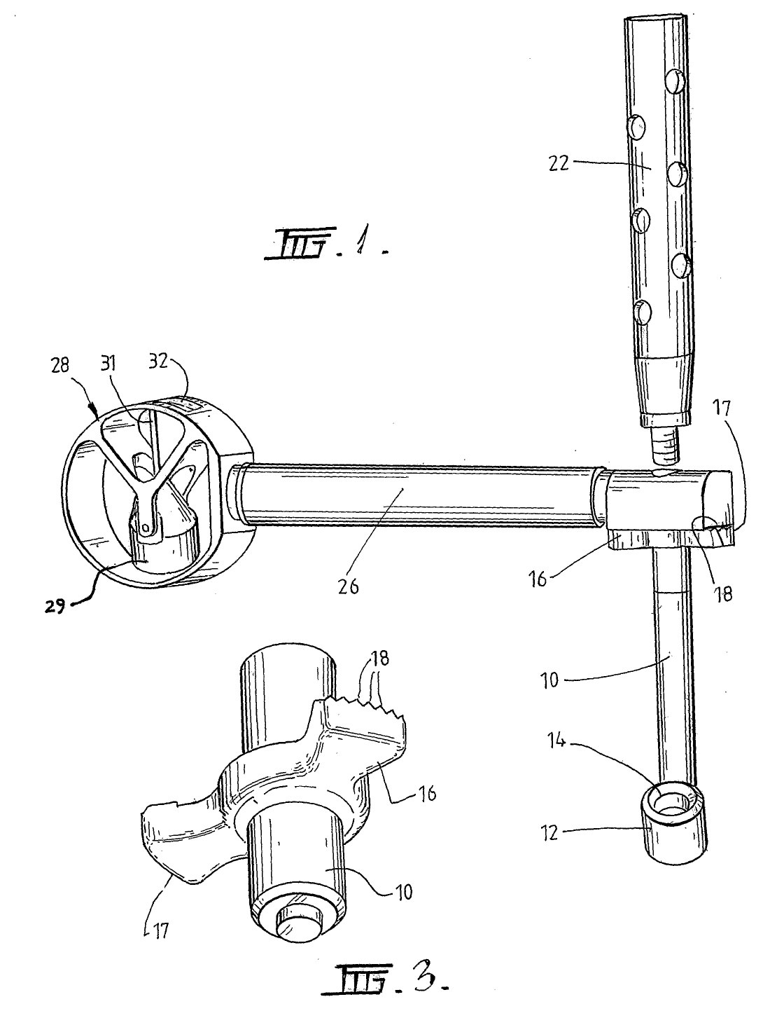

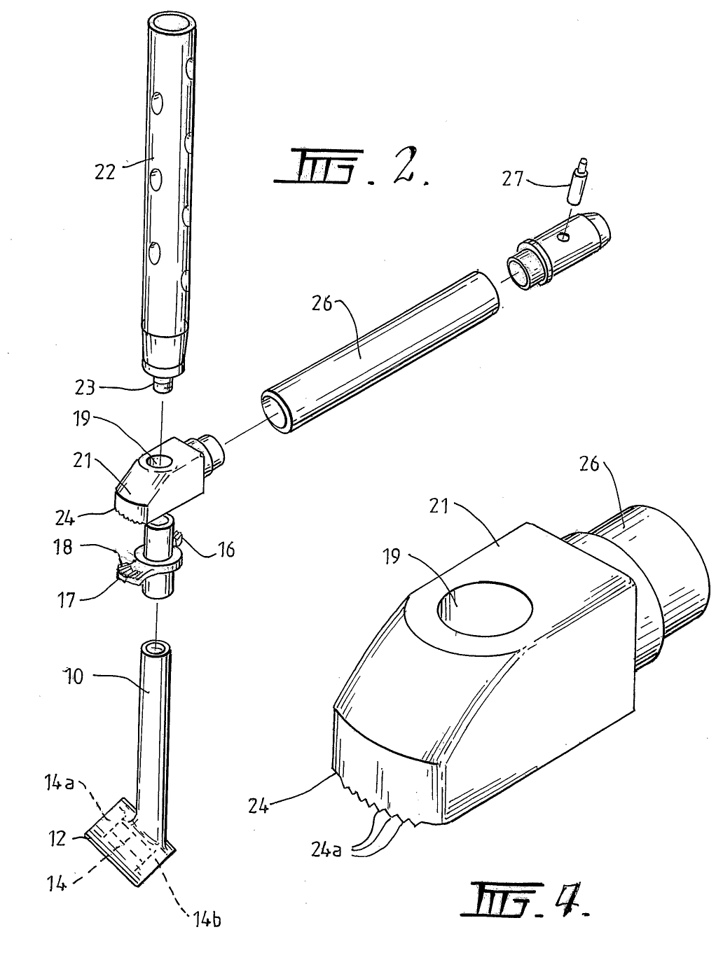

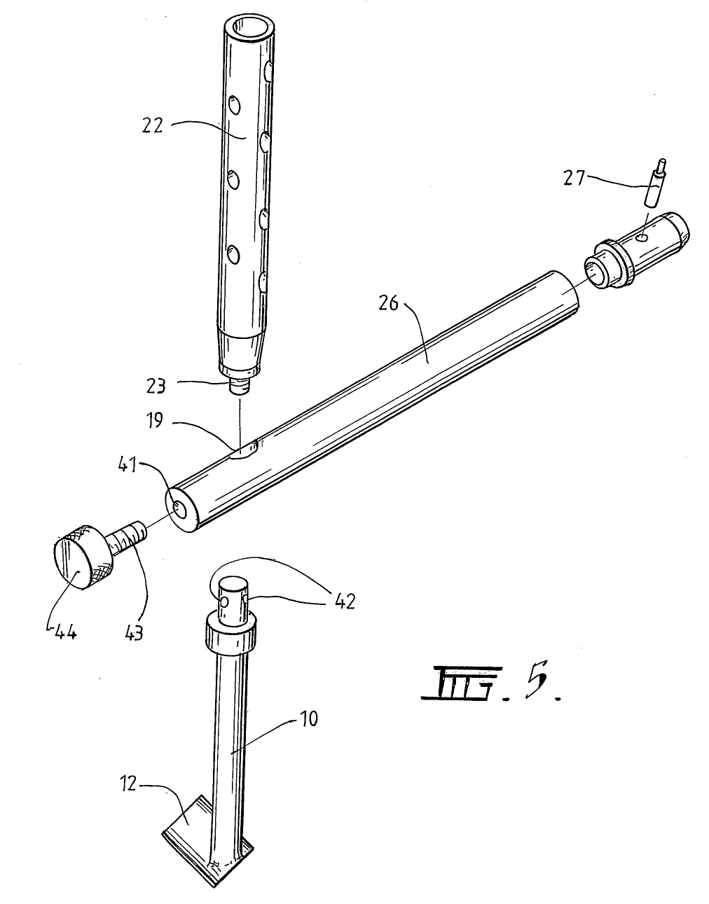

Referring to FIGS. 1 to 4, the surgical alignment guide of this embodiment has a shaft 10 carrying a prosthetic acetabular cup support boss 12 at its lower end. The boss 12 has a bore 14 the axis of which extends at an angle of 45° to the axis of the shaft 10. The bore 14 has larger diameter portions 14a and 14b at each end to facilitate engagement of a prosthetic acetabular cup spacer and a connector, such as a screw, with a prosthetic acetabular cup to be mounted to the support.

A pair of generally opposed, radially extending fingers 16 and 17 are mounted on the upper end of the shaft 10, spaced from the cup support boss 12. The fingers 16 and 17 are angularly spaced about the shaft axis such that radial lines defining the centre of each finger describe an angle of 150°. Each finger 16 and 17 is formed with a plurality of adjacent, radial groves defining teeth 18 which face in a direction away from the cup support boss 12.

A body element 21 has an opening 19 to receive the upper end of the shaft 10, and a coaxial handle 22 is assembled to the upper end of the shaft 10 by a screw thread 23. A radial surface segment 24 of the body element 21, which faces in the direction of the cup support boss 12, is formed with a plurality of complementary groves defining complementary teeth 24a adapted to mate with the teeth 18 of the fingers 16 and 17 such that the teeth 18 of upper toothed surface of one of the fingers 16 and 17 interengage the toothed surface 24 of the body element 21.

In use, the handle 22 may be loosened from its threaded engagement with the upper end of the shaft 10 to facilitate rotational movement of the shaft relative to the body element 21 whereby one or other of the fingers 16 and 17 can selectively be aligned so that the teeth 18 thereon engage the complimentary teeth 24a on the body element 21.

A plane containing the shaft axis and bore axis bisects the angle between the radial centre lines of the fingers 16 and 17. Therefore, when one of the fingers 16 is centrally engaged with the complimentary teeth 24a, the plane containing the axis of the shaft 10 and bore 14 extends at an angle of 15° to a shaft axial plane at right angles to the radial centre line of the finger 16.

Similarly, if the shaft 10 is rotated so that the finger 17 centrally engages with the complimentary teeth 24a of the body element 21, the plane containing the shaft and bore axis extends at 15° to an axial plane through the shaft 10 at right angles to the radial centre line of the finger 17.

Thus, the boss 12 and shaft 10 are able to be accurately positioned for both left hand and right hand acetabular cup insertion with the correct angular orientation. If, however, the anteversion needs to be modified for a particular patient, the shaft 10 may be rotated slightly relative to the body element 21 so that the teeth 18 engage others of the complimentary teeth 24 to thereby vary the angle of the plane containing the shaft and bore axis. The angle may be varied from between about 10° and about 20°, or more for each of the fingers 16 and 17.

The body element 21 has an arm 26 extending from the end thereof opposite the complimentary teeth 24a. The outer end of the arm 26 has a pair of opposed bayonet pins 27 one of which is of a larger diameter than the other. An alignment gauge 28 (FIG. 1) has a bayonet socket (not shown) to engage with the end of the arm 26 and the pins 27. The bayonet socket includes opposed J shaped slots of different widths so that the alignment gauge 28 can be fitted in only one orientation.

In the embodiment illustrated, the alignment gauge 28 is in the form of a plumb bob 29 having an upwardly extending needle 31 moveable within an opening 32. The plumb bob 29 is movable in at least two dimensions so that the needle 31 provides a visual guide to a surgeon as to the orientation of the arm 26 and the connected prosthetic acetabular cup support. With the arm 26 horizontal and the shaft 10 vertical, the plumb bob needle 31 is centrally located in the opening 32. Any movement away from the horizontal/vertical alignment is indicated by relative movement of the needle 31 in the opening 32 so that a surgeon is easily able to correctly align a prosthetic acetabular cup carried by the support for either a left hand or right hand hip replacement or revision surgery.

Referring to FIG. 5, a second embodiment of the invention is illustrated wherein the shaft 10 is formed adjacent its upper end with a pair of detents 42 in the outer cylindrical surface of the shaft 10. The arm 26 has an axially extended, threaded hole 41 extending from the end of the arm 26 into the opening 19 through which the upper end of the shaft 10 engages. A finger bolt 43 having a knurled head 44 is engaged in the threaded hole 41. The handle 22 engages the end of the shaft 10 to lock the shaft 10 within the opening 19.

The angular position of the boss 12 is set by engaging the end of the finger bolt 43 in one or other of the detents 42. The detents are angularly spaced 150° so that, when the inner end of the finger bolt 43 is engaged in one of the detents, the plane containing the axis of the shaft 10 and bore 14 extends at 15° to a plane containing the shaft axis which is at right angles to the axis of the arm 26 and finger bolt 43.

With this arrangement, the prosthetic acetabular cup support is able to be positioned for both left hand and right hand hip replacement or revision surgery by a simple adjustment, and when so adjusted, the bore 14 extends at the exact normal anteversion and abduction angle.

It will be appreciated that an alignment gauge 28, as described with reference to the first embodiment, may be engaged with the arm 26 of the second embodiment. Such an alignment gauge may be a plumb bob 29 or other form of alignment gauge to indicate the alignment position of the shaft 10 and arm 26.

It will also be appreciated that other forms of adjustment and securing the shaft 10 relative to the arm 26 may be used in the performance of the invention.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A surgical alignment guide for hip replacement or revision surgery comprising a prosthetic acetabular cup support connected to one end of an elongated carrier, said support having a bore to receive a connector to releasably secure a prosthetic acetabular cup thereto in a predetermined orientation, said bore extending at a predetermined angle relative to the carrier, an arm extending from the carrier at a position spaced from the support, and adjustment means associated with the elongated carrier whereby the angular position of the bore is variable with respect to the arm.

2. A surgical alignment guide according to claim 1 wherein the elongated carrier comprises a rod or shaft having the cup support fixed to one end thereof.

3. A surgical alignment guide according to claim 2 wherein the cup support comprises a boss fixed to the end of the rod or shaft, as by welding, so as to extend at an angle of 45° to the shaft axis.

4. A surgical alignment guide according to claim 1 wherein the axial bore through the boss has an enlarged diameter at least one end thereof.

5. A surgical alignment guide according to claim 4 wherein the enlarged bore is at a lower end of the cup support boss and is adapted to receive a spacer of a predetermined thickness to facilitate correct engagement of different sizes of prosthetic acetabular cups to the cup support boss.

6. A surgical alignment guide according to claim 1 wherein the elongated carrier includes a handle portion at the end spaced from the cup support.

7. A surgical alignment guide according to claim 6 wherein the handle portion is tubular and coaxial with the elongated carrier, and is mounted thereto by a threaded connection.

8. A surgical alignment guide according to claim 1 wherein the elongated carrier engages through an opening in the arm, at one end thereof, and is rotatable about its axis relative to the arm.

9. A surgical alignment guide according to claim 1 wherein a locking screw is provided in one end of the arm, the locking screw being adapted to engage within one of at least two detents formed in the elongated carrier such that the engagement of the locking screw with said one or other of the detents fixes the relative angular orientation of the cup support bore in relation to the arm.

10. A surgical alignment guide according to claim 1 wherein an outer end of the arm is adapted to be connected to a gauge which provides an alignment orientation indication of the cup support and a prosthetic acetabular cup attached thereto.

11. A surgical alignment guide according to claim 10 wherein the gauge is a plumb bob gauge which provides the appropriate indication of angular orientation.

12. A surgical alignment guide according to claim 1 wherein the adjustment means associated with the elongated carrier comprises two radial fingers extending from the elongated carrier.

13. A surgical alignment guide according to claim 12 wherein the fingers are provided with teeth on a radial surface, the teeth being adapted to engage corresponding teeth associated with the arm whereby the angular orientation of the bore is able to be adjusted through a range of different positions by varying the engagement of the respective teeth.

14. A surgical alignment guide according to claim 12 wherein the fingers are angularly spaced about the elongated carrier axis such that radial lines defining the centre of each finger describe an angle of 150°.

15. A surgical alignment guide according to claim 12 wherein each finger is formed with a plurality of adjacent, radial groves defining teeth which face in a direction away from the cup support.

16. A surgical alignment guide according to claim 12 wherein the arm includes a body element having an opening to receive an upper end of the elongated carrier, and a coaxial handle is assembled to the upper end of the elongated carrier by a screw thread.

17. A surgical alignment guide according to claim 16 wherein the body element, is formed with a radial surface segment which faces in the direction of the cup support and is formed with a plurality of complementary groves defining complementary teeth adapted to mate with teeth of the fingers such that the interengaged teeth define the angular orientation of the elongated carrier with respect to the arm.

18. A surgical alignment guide according to claim 17 wherein the handle is able to be loosened from its threaded engagement with the upper end of the elongated carrier to facilitate rotational movement of the elongated carrier relative to the body element whereby one or other of the fingers is selectively be aligned so that teeth thereon engage the complimentary teeth on the body element.

19. A surgical alignment guide according claim 12 wherein a plane containing the elongated carrier axis and bore axis bisects the angle between the radial centre lines of the fingers so that when one of the fingers is centrally engaged with the complimentary teeth of the body element the plane containing the axis of the elongated carrier and bore extends at an angle of 15° to a elongated carrier axial plane at right angles to the radial centre line of the respective finger.

20. A surgical alignment guide according to claim 19 wherein the elongated carrier may be rotated slightly relative to the body element so that the teeth of the finger engage with other complimentary teeth of the body element to thereby vary the angle of the plane containing the elongated carrier and bore axis, the angle being varied from between about 10° and about 20° for each of the fingers.

21. (canceled)
